(19) United States
(12) Patent Application Publication
(10) Pub. No.: US 2009/0053317 A1
(43) Pub. Date: Feb. 26, 2009

(54) MICROPARTICULATE SYSTEMS FOR THE ORAL ADMINISTRATION OF BIOLOGICALLY ACTIVE SUBSTANCES

(76) Inventors: Daniele Vigo, Santa Giulietta (Pavia) (IT); Massimo Faustini, Milano (IT); Sarah Scocca, Milano (IT); Eleonora Munari, Milano (IT); Maria Luisa Torre, Pavia (IT); Ubaldo Conte, Busto Arsizio (Varese) (IT); Francesco De Simone, Ogliara (Salemo) (IT); Rita Patrizia Aquino, Avellino (IT); Maria Rosaria Lauro, Nocera Inferiore (Salemo) (IT)

Correspondence Address: Steinfl & Bruno, 301 N Lake Ave Ste 810, Pasadena, CA, 91101, US

(21) Appl. No.: 12/158,044

(22) PCT Filed: Dec. 22, 2006
(86) PCT No.: PCT/IT2006/000874

§ 371 Date: Oct. 9, 2008

(30) Foreign Application Priority Data
Dec. 22, 2005 (IT) MI2005A002461

(51) Int. Cl. A61K 9/14; A61K 31/715; A61K 38/43; A61K 38/00

(52) U.S. Cl. 424/499; 424/489; 514/54; 424/94.1; 514/2

(57) ABSTRACT

The present invention relates to gastroresistant and enterosoluble microparticulate systems for the encapsulation of biologically active substances selected from: flavonoids, vitamins, antioxidants, immunostimulants, starchy and non-starchy polysaccharides, probiotics, prebiotics, intestinal trophism regulators, oligoelements, enzymes and bioactive peptides. Such microparticulate systems allow the administration of the aforementioned nutraceutic substances to animals such as porcines, bovines, caprines, ovines, equids, canids, felines, camelids, lagomorphs, rodents, fowl, and other mammals, including humans, fish and crustaceans, increasing the bioavailability.

### FIELD OF THE INVENTION

The present invention relates to microparticulate systems for the oral administration of biologically active substances such as hutraceutics and the relevant process for the preparation thereof.

### PRIOR ART

A “functional food” is defined as a foodstuff or a constituent thereof with positive effects on one or more specific body functions, going beyond pure nutritional effects, resulting in the improvement of the state of health or wellbeing and/or the prevention and treatment of diseases. A product with a defined chemical structure present as a natural constituent in a functional food is defined as a “nutraceutic”.

Functional foods or nutraceutics, including foodstuffs as they are or enriched, have potential health benefits if they are taken in efficacious doses and made bioavailable, resulting in their biological activities.

The use of nutraceutics and functional foods has been widespread among humans, but their rational use in the feedstuff industry is spreading in the zootechnical and veterinary fields as a consequence of the progressive reduction of the use of traditional drugs.

The nutraceutics used in the feedstuff industry for the manufacture of feeds for swine, bovids, caprines, ovines, equids, canids, felines, camelids, lagomorphs, rodents, fowl, and other mammals, fish and crustaceans are derived from a number of categories, such as flavonoids, vitamins, antioxidants, immune system stimulants, starchy and non-starchy polysaccharides, probiotics, prebiotics, intestinal trophism regulators, oligoelements, enzymes and bioactive peptides.

Said nutraceutics are characterised by considerable instability and are sensitive to environmental and biological factors, such as the gastric digestive processes, which result in significant loss of activity. At present, said nutraceutics are supplemented to feedstuffs in suitable doses, following conventional technological processes, which do not foresee the protection of the nutraceutic from the external environment.

Indeed, one of the limitations in the use of vitamins and antioxidants is their reduced stability in acidic environments, in the presence of oxygen or other oxidising agents. This instability has frequently lead to contradictory results in relation to their effective efficacy, and to their use at extremely high doses.

The use of vitamins, such as tocopherol, as nutraceutic substances has recently been proposed for improving the quality of meat, the stability of the muscle fibrils, enhanced tenderness, palatability, aroma and flavour: in bovids they ensure high levels of oxymyoglobin, with an enhanced, vibrant red colouration of the meat, even during storage, particularly appreciated by the market; in swine, they reduce lipid peroxidation and levels of malonaldehyde, an indicator of lipid peroxidation

From data in the literature, it appears that the use of vitamins and nutraceutic substances, if appropriately administered, result in improved meat quality in rabbits, in broilers and in fish such as carp.

The administration of high doses of tocopherol to various bovine species improves reproductive performance by regularising oestrous cycles, and reducing the incidence of placental retention and mastitis in lactating bovids.

Other nutraceutics that can be used in zootechnics belong to the flavonoid class. This class of molecules is recognised to have gastroprotective, antibacterial, antinflammatory and immunostimulatory activities, following the induction of the production of interferons. Other effects that can be ascribed to flavonoids include antioxidant and cell membrane protection activities, bronchodilatory and opioid effects, with the modulation of gastrointestinal activity and electrolyte flow across the gastrointestinal mucosa (antidiarrhoic effect). One of the problems associated with the use of flavonoids is their reduced bioavailability due to irregular absorption following oral administration.

There are numerous still unresolved problems which beset the rearing of both swine and bovids, and in many cases solutions for overcoming the diseases which can affect the entire stock or individual species, with serious financial losses for the farmer, have still to be found.

One of the major health problems currently affecting many farms includes gastro-oesophageal ulcers in swine during growth and fattening. In the majority of animals affected, the outcome of this disease is the death of the animal. Death due to ulcers can also be observed (even if less common) during weaning and among sows. In fatstock keeping, the affected animals appear pale and grow much slower. Many of them don't die and the ulcer has a tendency to heal over time, but growth is compromised, with obvious, negative financial repercussions for the farmer.

Another significant disease affecting swine species, just like the young of many other animal species, including humans, is diarrhea caused by rotavirus. Rotavirus infections in piglets have an incubation period of 2-4 days, depending on the virulence of the viral strain, the age of the piglets, the immune status of the sow, and the environmental and husbandry conditions. Under natural conditions, diarrhea can also manifest itself in newborn animals, but is more frequent in animals 2-6 weeks old, towards the end of suckling or in the first days post-weaning. Affected piglets become anorexic and depressed a few hours prior to the onset of diarrhea. Often, there is vomiting, but this is not a classic rotavirus symptom. Generally, affected adults do not show any symptoms, even though diarrhea is often observed in gilts. The diarrhea can be serious, and starts with watery or creamy faeces rapidly becoming liquid and profuse and yellowish or green in colour. The diarrhea can last for over ten days. The return to normality is gradual and can take 1-2 weeks. Dehydration is more evident in sucklers, and in those animals affected for longer periods of time. Morbidity usually exceeds 80%, affecting the entire farm in just a few days. Mortality can reach 20% and is higher in sucklers.

The use of nutraceutics in animal feed results in reduced use of antibiotics, anti-inflammatory drugs, painkillers and growth promoters.

The problem addressed by the present invention is that of improving the bioavailability of the nutraceutics administered to animals. As explained above, nutraceutics are currently added to fodder. That leads to the exposure of said substances to environmental factors, for example atmospheric oxygen, and biological factors, such as the gastric digestive processes. Said factors lead to the partial or total degradation of the nutraceutics added to fodder, and thus result in the poor bioavailability of said substances once administered to animals.

This problem is resolved by microparticulate systems and a process for the preparation thereof, as defined in the appended claims.

### DETAILED DESCRIPTION

It has now been unexpectedly found, and forms the subject of the present industrial invention, that the oral administration of suitably protected nutraceutic substances, as will be defined in detail hereinafter, improves the bioavailability of the nutraceutics themselves, avoiding the loss or drastic reduction of the activity thereof during the fodder preparation processes, during the storage of the same and during digestion and absorption in treated animals.

In particular, we have unexpectedly found that the use of suitably protected flavonoids as nutraceutics in species of swine results in a series of positive effects which are explained a variety of ways. The administration of microencapsulated flavonoids, as they are or vehicularised at a suitable concentration in fodder, has a protective effect on the gastrointestinal mucosa, preventing ulcers associated with antibacterial and inflammatory action. Furthermore, the stimulation of the immune system following increased release of interferons is obtained, along with an antioxidant and protective effect towards cellular membranes.

Furthermore, in swine species affected by pulmonary diseases, characterised by acute and chronic bronchospasm, the administration of suitably protected flavonoids as nutraceutics has a bronchodilatory effect.

Another effect unexpectedly found from the use of suitably protected flavonoids as nutraceutics in swine species is the “opioid” effect, characterised by the modulation of gastrointestinal motility (slowed transit) and the regulation of electrolyte flow through the intestinal mucosa. This mechanism results in marked antidiarrhetic activity, as will be further specified in the examples accompanying the present patent application.

One of the problems with the administration of flavonoids relates to their reduced bioavailability following oral administration.

Indeed, flavonoids can exist as aglycones or as glucosides (i.e. aglycones bound to a sugar). Generally, aglycones are poorly soluble and thus have very low dissolution rates in biological fluids. A low rate of dissolution implies irregular absorption and poor bioavailability. The glucoside is more soluble than the aglycone, and hence administration of the former results in increased bioavailability. Indeed, the aglycone is only absorbed following the hydrolysis, which is generally slow, of the glucoside sugar moiety by intestinal glucosidases, for absorption into peripheral circulation. The microencapsulation of flavonoids according to the present invention allows resolving the problem of their poor bioavailability, as they are delivered directly into the intestine, where they are released and degraded by intestinal glucosidases.

Other nutraceutic substances which may be encapsulated within the microparticulate systems of the present invention include: quercetin and rutin (quercetin conjugated to rhamnose and glucose).

Said substances are vehicularised in microparticulate systems constituted by polymers derived from cellulose such as cellulose acetophthalate, cellulose trimellitate with pH-dependent solubility, or methacrylic or ethylacrylate copolymers known as Eudragit E, L, S, RL, RS with pH-dependent solubility.

In particular, cellulose acetophthalate or copolymers of acrylic acid and ethyl acrylate, used in the preparation of microspheres and microparticulate or granulate systems, described and claimed in the present patent application, are insoluble in the acidic gastric environment, but very soluble in neutral-alkaline environments. Such formulations give more or less complete protection of the active substances in the gastric environment, and complete and rapid release of the drug, nutraceutic or active substance, as verified in in vitro tests, in simulated enteric environments.

Now we have unexpectedly found that the administration of microencapsulated flavonoids, according to the process claimed, allows avoidance of the onset of diseases and permits improved nutritional and productive efficiency with obvious advantageous repercussions for the health of animals treated, with undoubted financial advantages for the farmer.

Another aspect of the invention relates to the simultaneous supplementing of animal fodder with microcapsules containing nutraceutics, according to the invention, and non-encapsulated antioxidants. Hence, the purpose of the non-encapsulated antioxidants is to protect the fodder from environmental degradation and thus allow its long-term storage, while the encapsulated antioxidants are carried directly into the intestine, and released at that location.

The microparticulate systems of the present invention are constituted by a gastroresistant, biocompatible and biodegradable polymer matrix, comprising at least one gastroresistant and enterosoluble polymer, at least one monovalent divalent or trivalent metal ion salt of a biocompatible and biodegradable polymer having acid groups, at least one additional biocompatible and biodegradable polymer and biologically active substances.

By the term biologically active substances are meant nutraceutics i.e. flavonoids, vitamins, antioxidants, immunostimulants, starchy and non-starchy polysaccharides, probiotics, prebiotics, intestinal trophism regulators, oligoelements, enzymes and bioactive peptides.

Said microparticulate systems are used for the administration, preferably orally, of biologically active substances to animals selected from: porcines, bovines, caprines, ovines, equids, canids, felines, camelids, lagomorphs, rodents and other mammals, including humans, fowl, fish and crustaceans. Preferred animals are the young of such species.

The special composition of such microparticulate systems allows the protection of said biologically active substances from degradation by proteases and gastric acid, allowing their release into the intestine, where they may perform their activities.

Preferably, said at least one gastroresistant and enterosoluble polymer is selected from: phthalic acid cellulose esters, (for example: cellulose acetophthalate, hydroxypropyl-methylcellulose phthalate), trimellitic acid cellulose esters (for example: cellulose trimellitate, hydroxypropylcellulose trimellitate, hydroxypropyl-methylcellulose trimellitate); acrylates and polymethacrylates. Polymethacrylates are the most preferred.

Said at least one monovalent divalent or trivalent metal ion salt of a biocompatible and biodegradable polymer having acidic groups is a sodium, potassium, lithium, calcium, barium, strontium, zinc, aluminium, iron, or chromium salt of alginic acid, hyaluronic acid or xanthan gum.

Said at least one biocompatible and biodegradable polymer is selected from the group constituted by: glucans, scleroglucans, mannans, galactomannans, gellans, carrageenans, pectins, polyanhydrides, polyaminoacids, polyamines, xanthans, tragacanth gum, guar gum, xanthan gum, celluloses and derivatives thereof, carboxymethylcellulose, ethylcellulose, methylcellulose, hydroxypropylcellulose, hydroxyethylcellulose, hydroxypropylmethylcellulose, polyvinylalcohols, polyoxyethylenes, carboxyvinylpolymers, starches, collagens, chitins, chitosans, block copolymers of polyoxyethylene-polyoxypropylene block copolymers known as poloxamers.

Carboxymethylcellulose, ethylcellulose, methylcellulose, hydroxypropylcellulose, hydroxyethylcellulose hydroxypropylmethylcellulose, polyvinylalcohols, polyoxyethylenes, carboxyvinylpolymers, starches, collagens, chitins, chitosans, block copolymers of polyoxyethylene-polyoxypropylene block copolymers known as poloxamers are preferred.

The microparticulate systems described above and obtained by means of the process described below, have a diameter comprised of between 1 and 300 microns and preferably between 3 and 100 μm.

The present invention also relates to a process for the preparation of said gastroresistant microparticulate systems.

Said process comprises the following stages:
a) Preparing a solution, suspension or emulsion comprising at least one biocompatible and biodegradable polymer and one anionic, cationic, amphoteric or non-ionic surfactant;
b) Solubilising or dispersing at least one biologically active substance in the solution, suspension or emulsion from step a) (mixture A);
c) Preparing an aqueous solution of at least one monovalent metal ion salt of a biocompatible and biodegradable polymer (mixture B);
d) Adding solution B to mixture A;
e) preparing a solution or a dispersion of at least one gastroresistant and enterosoluble polymer (mixture C);
f) Adding mixture C to mixture B;
g) nebulising or extruding the solution or suspension from step f) into an aqueous solution of a soluble divalent or trivalent inorganic ion salt;

The presence of divalent or trivalent ions leads to the formation of an insoluble matrix constituted by, for example, the alginate salt of calcium and/or barium and/or some other divalent or trivalent cation. This leads to the formation of insoluble microparticulate systems containing the biologically active substances.

h) Alternatively, at step g), the mixture from step f) may be nebulised and dried using a spray-dryer, as known to those skilled in the art.

Mixture A obtained from step b) is preferably an emulsion or a suspension which is obtained by dissolving an equal or otherwise amount of a biocompatible-biodegradable polymer and an anionic, cationic, amphoteric or non-ionic surfactant in distilled water, preferably at room temperature. The quantities of the two aforementioned components are, respectively, comprised of between: 0.1% and 50% w/v, preferable between 0.4% and 30% w/v.

To the solution thus prepared is added the active substance, while stirring continuously until a stable solution, suspension or emulsion is obtained (mixture A). The quantity of active substance added is comprised of between 0.1% w/v and 50% w/v, preferably between 0.4% and 30% w/v.

Mixture C is a buffer solution at a pH comprised of between 5 and 9, but preferably between 7 and 8, and comprises at least one gastroresistant and enterosoluble polymer in a quantity of between 10% and 50% w/v, preferably between 5% and 25% w/v.

Mixture B is added to mixture A, in a preferred volumetric ratio of 1:2, and the solution thus obtained is added to mixture C in a preferred volumetric ratio of 3:1.

In step g), nebulisation takes place with the aid of orifices, nozzles, or syringes having sizes ranging from 10 μm to 5000 μm, preferably from 300 μm to 2000 μm. Extrusion takes place with the aid of automatic or semiautomatic microencapsulators, peristaltic or piston pumps or alternatives, or by means of a syringe, manually and/or automatically driven at such a speed as to produce from 10 to 250 drops/minute, preferably from 20 to 120 drops/minute.

Nebulisation or extrusion results in the formation of very small drops which are collected in an aqueous solution of a soluble divalent or trivalent inorganic ion salt, kept stirring at a speed of between 10 and 200 rpm, preferably between 20 and 100 rpm. The volumetric ratio between the extruded solution and the inorganic salt solution is between 1:1 and 1:6, preferably, the ratio is 1:4.

This divalent or trivalent ion inorganic salt is selected from: calcium, barium, strontium, zinc, aluminium, iron or chromium chloride, preferably calcium chloride, barium chloride or aluminium chloride. Even more preferably, it is calcium chloride. The concentration of said inorganic salt solutions is comprised of between 0.1 M and 2.0 M, preferably between 0.2 M and 0.8 M.

The presence of a divalent or trivalent metal salt leads to the formation of a matrix constituted by insoluble salts of the biodegradable and biocompatible polymer, having acidic groups, with the divalent or trivalent metal used, and thus the attainment of rapidly sedimenting microparticulate systems.

Such microparticulate systems have a spherical shape and are insoluble. They are separated from the solution by aspiration or filtration. Optionally, they may be washed several times with physiological solution (isotonic saline).

In one preferred aspect, the microparticulate systems thus obtained may be subjected to outer surface cross-linking, by means of interfacial polymerisation of the biocompatible and biodegradable polymer divalent or trivalent metal ion salt, using polyamine-type cross-linking agents such as, for example: protamine sulphate or phosphate, poly-L-lysine hydrobromide (molecular weight range from 1,000 Da to 80,0000 Da), polyvinylamine, chitosans (molecular weight range from 15,000 Da to 1,000,000 Da). Said cross-linking agents are preferably used as aqueous solutions at concentrations comprised of between 0.01% and 5% w/v.

The cross-linking reaction is carried out at a temperature comprised of between 5 and 40° C., preferably around 25° C. for periods of time comprised of between 1 minute and 120 minutes, preferably between 3 and 30 minutes.

The cross-linking reaction leads to the hardening of the membrane of the microparticulate systems, making them easier to handle.

In one preferred aspect, said microparticulate systems may be subsequently subjected to lyophilisation, using techniques known to those skilled in the art, or dried by means of any method known in the art which is not prejudicial to the activity of the encapsulated biologically active substance.

Alternatively, the production process for the microparticles of the invention may envisage the formation of a spheroidal granulate. In this case, thickeners, for example corn starch, lactose etc., and a biocompatible and biodegradable polymer, are added to the mixture of components. The wet mass thus obtained is extruded by means of a suitable granulator, as known in the art. Thus spheroidal granules, with a granulometric distribution comprised of between 50 and 1000 microns, and preferably between 150 and 500 microns, are obtained. Said granulate is then coated with a gastroresistant and enterosoluble polymer to give the microparticles of the invention.

Said microparticulate systems may be stored at temperatures comprised of between −20° C. and 40° C., preferably between 4° C. and 40° C., possibly in a controlled atmosphere, as known to those skilled in the art.

The microparticulate systems forming the subject of the present invention may be administered orally, by administration with a liquid diet or as supplements in solid feed.

In one additional aspect, the present invention relates to pre-packed feed for animals, with the microparticulate systems of the invention added, and feeds thus supplemented to which non-encapsulated antioxidants are also added.

This invention provides microparticulate systems having such dimensions as to allow optimal dispersion in solid and liquid foodstuffs without any problems involving the particles aggregating and, hence, separating out from solids or precipitating out of liquids. This allows easy administration to animals.

The gastroresistant microparticulate systems of the invention may be administered orally and afford, in acidic gastric environments, effective protection of the biologically active substances vehicularised, and the rapid release of the aforesaid substances, with high biological activity, in the enteric environment (small or large intestine).

Said gastroresistant microparticulate systems have significant application potential in the sector of veterinary gastroenterology and nutrition, especially in monogastric animal species, but also in polygastric non-ruminants and in those ruminants with still non-functional pre-stomachs.

Such preparations may be classified among the zootechnical feed additives (as described in the 1^{st} enclosure to Reg. CE No 1831/2003).

In livestock farming, the invention resolves the essential problem of the administration of active nutraceutic substances in quantities sufficient to allow their beneficial effects to be manifest.

### EXAMPLE 1

### Preparation of Microparticulate Systems Containing 6% α-Tocopherol

Emulsion A: Identical quantities of Poloxamer (0.4% w/v BASF, Ludwigshafen, Germany) and sodium lauryl sulphate (0.4% w/v Sigma-Aldrich, Milan, Italy) are dissolved in distilled water at room temperature. To the resulting solution is added, with constant stirring, alpha-tocopherol (1.6% w/v, Sigma-Aldrich, Milan, Italy) to give a stable and homogeneous emulsion.

Solution B: A 2% aqueous solution of low viscosity sodium alginate (250 cps, 2% solution, 25° C.) (Sigma-Aldrich, Milan, Italy) is prepared at room temperature.

Solution C: A 20% w/v solution of polymethacrylate (Eudragit S100®, Röhm Pharma, GmbH, Darmstadt, Germany) in phosphate buffer pH 7.5.

Solution C is added to solution B, in a volumetric ratio of 1:2, with constant stirring, and said solution is added to emulsion A, in a volumetric ratio of 3:1, again with constant stirring.

The percentage composition of the resulting emulsion is:
5% Polymethacrylate
1% Sodium alginate
0.1% Poloxamer
0.1% Sodium lauryl sulphate
0.4% Alpha-Tocopherol

Using a peristaltic pump, the resulting emulsion is nebulised by means of a spray-dryer (Büchi Mini Spray Dryer) fitted with a 0.5 mm diameter nozzle, with an air inlet temperature of 120° C., outlet temperature of 100° C., and an applied pressure of 4 atm.

Microparticulate systems are obtained, which are then suitably harvested, as known to those skilled in the art. Said microparticulate systems appear as a fine powder, insoluble in water, with a granulometric distribution comprised of between 5 and 35 microns and with good wettability, flow and fluidity properties.

Determination of the quantity of alpha-tocopherol contained has been performed spectrophotometrically at 291 nm, following dissolution of the microparticulate systems in absolute ethanol. The titre is 101±3% with respect to the theoretical value.

The microparticulate systems are subsequently subjected to assay as prescribed in the Pharmacopeia (FUI XI) for gastroresistant pharmaceutical forms, so as to assess the in vitro stability of the vitamin in acidic environments, and release in simulated enteric environments.

### EXAMPLE 2

### Preparation of Microparticulate Systems Containing 22% α-Tocopherol

Emulsion A

Poloxamer 407 (2% w/v, BASF, Ludwigshafen, Germany) and sodium lauryl sulphate (2% w/v, Sigma-Aldrich, Milan, Italy) are dissolved in distilled water at room temperature with constant magnetic stirring at 100 rpm. Alpha-tocopherol (8% w/v, Sigma-Aldrich, Milan, Italy) is added to the solution with turbine stirring (Ultra Turrax) for 15 minutes: a stable emulsion is obtained.

Solution B:

An aqueous solution of low viscosity sodium alginate (250 cps, 2% solution, 25° C.) (alginic acid, sodium salt, low viscosity, Sigma-Aldrich, Milan, Italy) at a concentration of 2% w/v, is prepared with constant magnetic stirring at 100 rpm at room temperature.

Solution C

A 20% w/v solution of polymethacrylate (Eudragit S100®, Röhm Pharma, Darmstadt, Germany) in phosphate buffer at pH 7.5 is prepared by stirring at room temperature.

Solution C is added to solution B, in a volumetric ratio of 1:2, with constant magnetic stirring, and said solution is added to emulsion A, in a volumetric ratio of 3:1, with constant turbine stirring for 15 minutes. Using a peristaltic pump, the resulting emulsion is nebulised by means of a spray-dryer (Büchi Mini Spray Dryer) fitted with a 0.5 mm diameter nozzle, with an air inlet temperature of 120° C., outlet temperature of 100° C., and an applied pressure of 4 atm.

Microparticulate systems are obtained, which are then suitably harvested, as known to those skilled in the art.

The products appear as fine powders with good flow and fluidity properties.

The composition of the product, calculated from the composition of the nebulised solution, is as follows:
55.56% Polymethacrylate
11.11 Sodium alginate
5.56% Poloxamer
5.56% Sodium lauryl sulphate
22.22% Alpha-tocopherol

The microparticulate systems, which are insoluble in water, are characterised by normal granulometric distribution and mean diameter of 19±12.8 microns, as determined by means of laser scattering (Coulter LS230, Beckman-Coulter, Fullerton, Calif., USA).

The powder has good free-flow and wettability properties, and is hence particularly suitable to be added to solid and liquid feeds, in order to obtain homogeneous mixtures or suspensions.

Following the preparation of the microparticulate systems, determination of the quantity of alpha-tocopherol contained in the microparticulate systems has been determined spectrophotometrically at a wavelength of 291 nm following dissolution of the microparticulate systems in absolute ethanol. The titre is equal to 102.0±1.84% (n=4).

The microparticulate systems have been subjected to assay as prescribed in the Pharmacopeia (FUI XI) for gastroresistant pharmaceutical forms, as reported in detail above, so as to assess the in vitro stability of the vitamin in acidic environments, and release in simulated enteric environments.

### EXAMPLE 3

### Preparation of Microparticulate Systems Containing 6% Rutin

Suspension A:

Poloxamer 407 (0.4% w/v, BASF, Ludwigshafen, Germany) and sodium lauryl sulphate (0.4% w/v, Sigma-Aldrich, Milan, Italy) are dissolved in distilled water at room temperature with constant magnetic stirring at 100 rpm. Rutin (1.6% w/v, Sigma-Aldrich, Milan, Italy) is added to the solution with turbine stirring (Ultra Turrax) for 15 minutes: a sable suspension is obtained.

Solution B:

An aqueous solution of low viscosity sodium alginate (250 cps, 2% solution, 25° C.) (alginic acid, sodium salt, low viscosity, Sigma-Aldrich, Milan, Italy) at a concentration of 2% w/v, is prepared with constant magnetic stirring at 100 rpm at room temperature.

Solution C:

A 20% w/v solution of polymethacrylate (Eudragit S100®, Röhm Pharma, Darmstadt, Germany) in phosphate buffer at pH 7.5 is prepared by stirring at room temperature.

Solution C is added to solution B, in a volumetric ratio of 1:2, with constant magnetic stirring; said solution is added to suspension A, in a volumetric ratio of 3:1, with constant turbine stirring (Ultra Turrax) for 15 minutes. Using a peristaltic pump, the resulting emulsion is nebulised by means of a spray-dryer (Büchi Mini Spray Dryer) fitted with a 0.5 mm diameter nozzle, with an air inlet temperature of 120° C., outlet temperature of 100° C., and an applied pressure of 4 atm.

Microparticulate systems are obtained, which are then suitably harvested, as known to those skilled in the art.

The products appear as fine powders with good flow and fluidity properties.

The composition of the product, calculated from the composition of the nebulised solution, is as follows:
75.76% Polymethacrylate
15.15% Sodium alginate
1.52% Poloxamer
1.52% Sodium lauryl sulphate
6.06% Rutin

The microparticulate systems, which are insoluble in water, are characterised by normal granulometric distribution and mean diameter of 21.9±13.7 microns, as determined by means of laser scattering (Coulter LS230, Beckman-Coulter Inc., Fullerton, Calif., USA).

The powder has good free-flow and wettability properties, and is hence particularly suitable to be added to solid and liquid feeds, in order to obtain homogeneous mixtures or suspensions.

Following the preparation of the microparticulate systems, determination of the quantity of rutin contained in the microparticulate systems has been determined spectrophotometrically at a wavelength of 367 nm following dissolution of the microparticulate systems in phosphate buffer at pH 7.5. The titre is equal to 108.0±1.84% (n=4) with respect to the theoretical value.

The microparticulate systems have subsequently been subjected to assay as prescribed in the Pharmacopoeia (FUI XI) for gastroresistant pharmaceutical forms, reported in detail above, so as to assess the in vitro stability of the rutin.

In particular, in simulated acidic environments, pH 1.0, less than 20% of the active substance is released after 120 minutes, and with subsequent switching to pH 7.5, the complete release of the active substance vehicularised in the microparticulate systems is obtained within 15 minutes.

### EXAMPLE 4

### Preparation of Microparticulate Systems Containing 11% Rutin

Suspension A:

Poloxamer 407 (0.8% w/v, BASF, Ludwigshafen, Germany) and sodium lauryl sulphate (0.8% w/v, Sigma-Aldrich, Milan, Italy) are dissolved in distilled water at room temperature with constant magnetic stirring at 100 rpm. Rutin (3.2% w/v, Sigma-Aldrich, Milan, Italy) is added to the solution with turbine stirring (Ultra Turrax) for 15 minutes: a stable emulsion is obtained.

Solution B:

An aqueous solution of low viscosity sodium alginate (250 cps, 2% solution, 25° C.) (alginic acid, sodium salt, low viscosity, Sigma-Aldrich, Milan, Italy) at a concentration of 2% w/v, is prepared with constant magnetic stirring at 100 rpm at room temperature.

Solution C:

A 20% w/v solution of polymethacrylate (Eudragit S100®, Röhm Pharma, Darmstadt, Germany) in phosphate buffer at pH 7.5 is prepared by stirring at room temperature.

Solution C is added to solution B, in a volumetric ratio of 1:2, with constant magnetic stirring; said solution is added to emulsion A, in a volumetric ratio of 3:1, with constant turbine stirring for 15 minutes.

Using a peristaltic pump, the resulting suspension is nebulised by means of a spray-dryer (Büchi Mini Spray Dryer) fitted with a 0.5 mm diameter nozzle, with an air inlet temperature of 120° C., outlet temperature of 100° C., and an applied pressure of 4 atm.

Microparticulate systems are obtained, which are then suitably harvested, as known to those skilled in the art.

The products appear as fine powders with good flow and fluidity properties.

The composition of the product, calculated from the composition of the nebulised solution, is as follows:
69.44% Polymethacrylate
13.89% Sodium alginate
2.78% Poloxamer
2.78% Sodium lauryl sulphate
11.11% Rutin

The microparticulate systems, which are insoluble in water, are characterised by normal granulometric distribution and mean diameter of 23.8±14.3 microns, as determined by means of laser scattering (Coulter LS230, Beckman-Coulter Inc., Fullerton, Calif., USA).

The powder has good free-flow and wettability properties, and is hence particularly suitable to be added to solid and liquid feeds, in order to obtain homogeneous mixtures or suspensions.

Following the preparation of the microparticulate systems, determination of the quantity of rutin contained in the microparticulate systems has been determined spectrophotometrically at a wavelength of 367 nm following dissolution of the microparticulate systems in phosphate buffer at pH 7.5. titre is equal to 91.43±10% (n=4) with respect to the theoretical value.

The microparticulate systems have been subjected to assay as prescribed in the Pharmacopeia (FUI XI) for gastroresistant pharmaceutical forms, as reported in detail above, so as to assess the in vitro stability of the rutin in acidic environments, and release in simulated enteric environments.

In particular, in simulated acidic environments, pH 1.0, less than 15% of the active substance is released after 120 minutes, and with subsequent switching to pH 7.5, the complete release of the active substance vehicularised in the microparticulate systems is obtained within 15 minutes.

### EXAMPLE 5

### Preparation of Microparticulate Systems Containing 22% Rutin

Suspension A

Poloxamer 407 (2% w/v, BASF, Ludwigshafen, Germany) and sodium lauryl sulphate (2% w/v, Sigma-Aldrich, Milan, Italy) are dissolved in distilled water at room temperature with constant magnetic stirring at 100 rpm. Rutin (8% w/v, Sigma-Aldrich, Milan, Italy) is added to the solution with turbine stirring (Ultra Turrax) for 15 minutes: a sable suspension is obtained.

Solution B:

An aqueous solution of low viscosity sodium alginate (250 cps, 2% solution, 25° C.) (alginic acid, sodium salt, low viscosity, Sigma-Aldrich, Milan, Italy) at a concentration of 2% w/v, is prepared with constant magnetic stirring at 100 rpm at room temperature.

Solution C:

A 206 w/v solution of polymethacrylate (Eudragit S100®, Röhm Pharma, GmbH, Darmstadt, D) in phosphate buffer at pH 7.5 is prepared by stirring at room temperature.

Solution C is added to solution B, in a volumetric ratio of 1:2, with constant magnetic stirring; said solution is added to emulsion A, in a volumetric ratio of 3:1, with constant turbine stirring for 15 minutes.

Using a peristaltic pump, the resulting suspension is nebulised by means of a spray-dryer (Büchi Mini Spray Dryer) fitted with a 0.5 mm diameter nozzle, with an air inlet temperature of 120° C., outlet temperature of 100° C., and an applied pressure of 4 atm.

Microparticulate systems are obtained, which are then suitably harvested, as known to those skilled in the art.

The product appears as a fine powder, with good flow and fluidity properties.

The composition of the product, calculated from the composition of the nebulised solution, is as follows:
55.56% Polymethacrylate
11.11% Sodium alginate
5.56% Poloxamer
5.56% Sodium lauryl sulphate
22.22% Rutin

The microparticulate system obtained is insoluble in water and is characterised by a normal granulometric distribution and a mean diameter of 21.2±12.7 microns, as determined by means of laser scattering (Coulter LS230, Beckman-Coulter, Fullerton, Calif., USA).

The powder has good free-flow and wettability properties, and is hence particularly suitable to be added to solid and liquid feeds, in order to obtain homogeneous mixtures or suspensions.

Following preparation of the microparticulate systems, determination of the quantity of rutin contained within the microparticulate systems has been performed by spectrophotometry at a wavelength of 367 nm following dissolution of the microparticulate systems in absolute ethanol. The titre has been equal to 102.4±2.6% (n=4).

The microparticulate systems obtained have subsequently been subjected to assay as prescribed in the Pharmacopoeia (FUI XI) for gastroresistant pharmaceutical forms, as reported above in detail, so as to assess the in vitro stability of the rutin in an acidic environment, and the release thereof in a simulated enteric environment.

In particular, in a simulated acidic environment, pH 1.0, less than 10% of the active substance is released within 120 minutes, and with subsequent switching to pH 7.5, the complete release of the active substance vehicularised within the microparticulate systems is obtained within 15 minutes.

### EXAMPLE 6

### Preparation of Microparticulate Systems Containing 6% Quercetin

Suspension A

Poloxamer 407 (0.4% w/v, BASF, Ludwigshafen, Germany) and sodium lauryl sulphate (0.4% w/v, Sigma-Aldrich, Milan, Italy) are dissolved in distilled water at room temperature with constant magnetic stirring at 100 rpm. Quercetin (1.6% w/v, Sigma-Aldrich, Milan, Italy) was added to the solution with turbine stirring (Ultra Turrax) for 15 minutes: a stable suspension is obtained.

Solution B:

An aqueous solution of low viscosity sodium alginate (250 cps, 2% solution, 25° C.) (alginic acid, sodium salt, low viscosity, Sigma-Aldrich, Milan, Italy) at a concentration of 2% w/v is prepared with magnetic stirring at 100 rpm at room temperature.

Solution C

A 20% w/v solution of polymethacrylate (Eudragit S100, Röhm Pharma, Darmstadt, Germany) in phosphate buffer at pH 7.5 is prepared at room temperature with constant stirring.

Solution C is added to solution B in a volumetric ratio of 1:2 and kept stirring using a magnetic stirrer; said solution is then added to suspension A in a volumetric ratio of 3:1 with constant turbine stirring (Ultra Turrax) for 15 minutes.

Using a peristaltic pump, the resulting solution is nebulised using a spray-dryer (Büchi Mini Spray Dryer) fitted with a 0.5 mm diameter nozzle, at an air input temperature of 120° C., output temperature of 100° C., with an applied pressure of 4 atm.

Microparticulate systems are obtained which are suitably harvested as known to those skilled in the art.

The products appear as a fine powder, with good flow and fluidity properties.

The composition of the product, calculated from the composition of the nebulised solution, is as follows:
75.76% Polymethacrylate
15.15% Sodium alginate
1.52% Poloxamer
1.52% Sodium lauryl sulphate
6.06% Quercetin

The microparticulate systems, which are insoluble in water, are characterised by a normal granulometric distribution and a mean diameter of 21.3±12.9 microns, as determined by means of laser scattering (Coulter LS230, Beckman-Coulter, Fullerton, Calif., USA).

The powder has good free-flow and wettability properties, and is hence particularly suitable to be added to solid and liquid feeds, in order to obtain homogeneous mixtures or suspensions.

Following preparation of the microparticulate systems, determination of the quantity of quercetin contained within the microparticulate systems has been performed by spectrophotometry at a wavelength of 366 nm following dissolution of the microparticulate systems in phosphate buffer at pH 7.5. The titre has been equal to 97.5±11.5% (n=4) with respect to the theoretical value.

The microparticulate systems have subsequently been subjected to assay as prescribed in the Pharmacopoeia (FUI XI) for gastroresistant pharmaceutical forms, as reported above in detail, so as to assess the in vitro stability of the quercetin in an acidic environment, and the release thereof in a simulated enteric environment.

In particular, in a simulated acidic environment, pH 1.0, less than 15% of the quercetin is released within 120 minutes, and with subsequent switching to pH 7.5, the release of no less than 50% of the active substance vehicularised within the microparticulate systems is obtained within 15-30 minutes.

### EXAMPLE 7

### Preparation of Microparticulate Systems Containing 11% Quercetin

Suspension A:

Poloxamer 407 (0.8% w/v, BASF, Ludwigshafen, Germany) and sodium lauryl sulphate (0.8% w/v, Sigma-Aldrich, Milan, Italy) are dissolved in distilled water at room temperature with constant magnetic stirring at 100 rpm. Quercetin (3.2% w/v, Sigma-Aldrich, Milan, Italy) was added to the solution with turbine stirring (Ultra Turrax) for 15 minutes: a stable suspension is obtained.

Solution B:

An aqueous solution of low viscosity sodium alginate (250 cps, 2% solution, 25° C.) (alginic acid, sodium salt, low viscosity, Sigma-Aldrich, Milan, Italy) at a concentration of 2% w/v is prepared with magnetic stirring at 100 rpm at room temperature.

Solution C:

A 20% w/v solution of polymethacrylate (Eudragit S100®, Röhm Pharma GmbH, Darmstadt, Germany) in phosphate buffer at pH 7.5 is prepared at room temperature with constant stirring.

Solution C is added to solution B in a volumetric ratio of 1:2 and kept stirring using a magnetic stirrer; said solution is then added to emulsion A in a volumetric ratio of 3:1 with constant turbine stirring (Ultra Turrax) for 15 minutes.

Using a peristaltic pump, the resulting solution is nebulised using a spray-dryer (Büchi Mini Spray Dryer) fitted with a 0.5 mm diameter nozzle, at an air input temperature of 120° C., output temperature of 100° C., with an applied pressure of 4 atm.

Microparticulate systems are obtained which are suitably harvested as known to those skilled in the art.

The product appears as a fine powder, with good flow and fluidity properties.

The composition of the product, calculated from the composition of the nebulised solution, is as follows:
69.44% Polymethacrylate
13.89% Sodium alginate
2.78% Poloxamer
2.78% Sodium lauryl sulphate
11.11% Quercetin

The microparticulate system obtained is insoluble in water and is characterised by a normal granulometric distribution and a mean diameter of 27.1±6.0 microns, as determined by means of laser scattering (Coulter LS230, Beckman-Coulter Inc., Fullerton, Calif., USA).

The powder has good free-flow and wettability properties, and is hence particularly suitable to be added to solid and liquid feeds, in order to obtain homogeneous mixtures or suspensions.

Following preparation, determination of the quantity of quercetin contained within the microparticulate systems has been performed by spectrophotometry at a wavelength of 366 nm following prior dissolution in phosphate buffer at pH 7.5. The titre has been equal to 100.7±15.8% (n=4) with respect to the theoretical value.

The microparticulate systems have subsequently been subjected to assay as prescribed in the Pharmacopoeia (FUI XI) for gastroresistant pharmaceutical forms, as reported above in detail, so as to assess the in vitro stability of the quercetin in an acidic environment, and the release thereof in a simulated enteric environment.

In particular, in a simulated acidic environment, pH 1.0, less than 5% of the quercetin is released within 120 minutes, and with subsequent switching to pH 7.5, the release of no less than 60% of the active substance vehicularised within the microparticulate systems is obtained within 15-30 minutes.

### EXAMPLE 8

### Preparation of Microparticulate Systems Containing 22% Quercetin

Suspension A:

Poloxamer 407 (2% W/v, BASF, Ludwigshafen, Germany) and sodium lauryl sulphate (2% w/v, Sigma-Aldrich, Milan, Italy) are dissolved in distilled water at room temperature with constant magnetic stirring at 100 rpm. Quercetin (8% w/v, Sigma-Aldrich, Milan, Italy) was added to the solution with turbine stirring (Ultra Turrax) for 15 minutes: a stable suspension is obtained.

Solution B:

An aqueous solution of low viscosity sodium alginate (250 cps, 2% solution, 25° C.) (alginic acid, sodium salt, low viscosity, Sigma-Aldrich, Milan, Italy) at a concentration of 2% w/v is prepared with magnetic stirring at 100 rpm at room temperature.

Solution C:

A 20% w/v solution of polymethacrylate (Eudragit S100®, Röhm Pharma GmbH, Darmstadt, Germany) in phosphate buffer at pH 7.5 is prepared at room temperature with constant stirring.

Solution C is added to solution B in a volumetric ratio of 1:2 and kept stirring using a magnetic stirrer, and said solution is then added to emulsion A in a volumetric ratio of 3:1 with constant turbine stirring (Ultra Turrax) for 15 minutes.

Using a peristaltic pump, the resulting solution is nebulised using a spray-dryer (Büchi Mini Spray Dryer) fitted with a 0.5 mm diameter nozzle, at an air input temperature of 120° C., output temperature of 100° C., with an applied pressure of 4 atm.

Microparticulate systems are obtained which are suitably harvested as known to those skilled in the art.

The product appears as a fine powder, with good flow and fluidity properties.

The composition of the product, calculated from the composition of the nebulised solution, is as follows:
55.56% Polymethacrylate
11.11% Sodium alginate
5.56% Poloxamer
5.56% Sodium lauryl sulphate
22.22% Quercetin

The microparticulate system obtained is insoluble in water and is characterised by a normal granulometric distribution and a mean diameter of 27.3 microns, as determined by means of laser scattering (Coulter LS230, Beckman-Coulter Inc., Fullerton, Calif., USA).

The powder has good free-flow and wettability properties, and is hence particularly suitable to be added to solid and liquid feeds, in order to obtain homogeneous mixtures or suspensions.

Following preparation of the microparticulate systems, determination of the quantity of quercetin contained within the microparticulate systems has been performed by spectrophotometry at a wavelength of 367 nm following prior dissolution of the microparticulate systems in absolute ethanol. The titre has been equal to 98.7% (n=4).

The microparticulate systems have subsequently been subjected to assay as prescribed in the Pharmacopoeia (FUI XI) for gastroresistant pharmaceutical forms, as reported above in detail, so as to assess the in vitro stability of the quercetin in an acidic environment, and the release thereof in a simulated enteric environment.

In particular, in a simulated acidic environment, pH 1.0, less than 10% of the quercetin is released within 120 minutes, and with subsequent switching to pH 7.5, the release of no less than 50% of the active substance vehicularised within the microparticulate systems is obtained within 15-30 minutes.

### EXAMPLE 9

### Preparation of a Rutin-Containing Granulate

Gastroresistant Granulate

Appropriate quantities of Poloxamer 407 (BASF, Ludwigshafen, Germany) and sodium lauryl sulphate (Sigma-Aldrich, Milan, Italy) are mixed in a suitable powder mixer, along with rutin (Sigma-Aldrich, Milan, Italy) and corn starch, lactose and other constituents, such as those known to those skilled in the art, are added to give a homogeneous mixture. Said mixture is imbibed using a binding solution consisting of a 10% aqueous solution of Polyvinylpyrrolidone (Kollidon 19-32 BASF). The wet mass is extruded through a suitable granulator-spheroniser to give a spheroidal granulate with a granulometric distribution comprised of between 50 and 1000 microns, and preferably between 150 and 500 microns.

Said spheronised granulate is coated, in a coating pan or in a fluidised bed by spraying a solution of cellulose acetophthalate (Sigma) or polymethacrylate (Eudragit S100®, Röhm Pharma GmbH, Darmstadt, Germany) in phosphate buffer at pH 7.5, supplemented with film plasticisers, such as those known to those skilled in the art.

The operation proceeds until the coating of the spheronised particles is complete and even.

The coated granulate has subsequently been subjected to assay as prescribed in the Pharmacopoeia (FUI XI) for gastroresistant pharmaceutical forms, as reported above in detail, so as to assess the in vitro stability of the rutin in an acidic environment, and the release thereof in a simulated enteric environment.

1. Microparticulate systems consisting of a gastroresistant, biocompatible and biodegradable polymer matrix containing biologically active substances wherein said matrix comprises:
at least one gastroresistant and enterosoluble polymer selected from: phthalic acid cellulose esters, trimellitic acid cellulose esters, acrylates and polymethacrylates
at least one anionic, cationic, amphoteric or non-ionic surfactant;
at least one monovalent, divalent or trivalent metal ion salt of a biocompatible and biodegradable polymer having acidic groups, selected from: salts of alginic acid, of hyaluronic acid and of xanthan gum;
at least one additional biocompatible and biodegradable polymer selected from: glucans, scleroglucans, mannans, galactomannans, gellans, carrageenans, pectins, polyanhydrides, polyaminoacids, polyamines, xanthans, tragacanth gum, guar gum, xanthan gum, celluloses and derivatives thereof, polyvinylalcohols, polyoxyethylenes, carboxyvinylpolymers, starches, collagens, chitins, chitosans, block copolymers of polyoxyethylene-polyoxypropylene known as poloxamers.
and wherein said biologically active substances are selected from:
flavonoids, vitamins, antioxidants, immunostimulants, starchy and non-starchy polysaccharides, probiotics, prebiotics, intestinal trophism regulators, oligoelements, enzymes and bioactive peptides.

2. The microparticulate systems according to claim 1 wherein said at least one gastroresistant and enterosoluble polymer is a polymethacrylate.

3. The microparticulate systems according to claim 1, wherein said monovalent divalent or trivalent metal ion salt of a biocompatible and biodegradable polymer having acidic groups is a sodium, potassium, lithium, calcium, barium, strontium, zinc, aluminium, iron, or a chromium salt of alginic acid, hyaluronic acid or xanthan gum.

4. The microparticulate systems according to claim 1, wherein said additional biocompatible and biodegradable polymer is a block copolymer of polyoxyethylene-polyoxypropylene known as poloxamers.

5. The microparticulate systems according to claim 1, having a diameter of between 1 and 300 microns.

6. A method for preparation of a gastroresistant pharmaceutical formulation, said formulation for administration of biologically active substances to animals, the method comprising:
mixing said biologically active substances with a gastroresistant, biocompatible and biodegradable polymer matrix to obtain the microparticulate systems according to claim 1 in a gastroresistant pharmaceutical formulation.

7. The method according to claim 6, wherein said animals include: porcines, bovines, caprines, ovines, equids, canids, felines, camelids, lagomorphs, rodents and other mammals including humans, fowl and fish.

8. The method according to claim 6, wherein said administration is performed by oral administration.

9. The method according to claim 8, wherein said microparticulate systems are combined with a liquid or solid diet or used as solid supplements for animal feed.

10. A method for preparation of a medicament for administration to animals according to claim 7, the method comprising:
mixing biologically active substances with a gastroresistant, biocompatible and biodegradable polymer matrix to obtain the microparticulate systems according to claim 1 in a medicament,
wherein said medicament is for prevention of ulcers; for stimulation of the immune system following increased release of interferons; for slowing gastrointestinal transit and motility with a regulatory effect on electrolyte flux across the intestinal mucosa of the animal and consequent antidiarrhoic activity for the animal; for reduced lipid peroxidation malonaldehyde content; for improved reproductive performance and regularising oestrous cycles; for reduced incidence of placental retention; for reduced incidence of mastitis; and/or for improving respiratory efficiency.

11. The method according to claim 10, for the preparation of a nutraceutic or a medicament having antibacterial, anti-inflammatory, antioxidant and cell membrane protective action, said nutraceutic or medicament for the treatment of lung disorders characterised by acute or chronic bronchospasm.

12. The method according to claim 10 wherein the medicament is for improving the quality and organoleptic characteristics of meat, even during storage.

13. Animal feeds supplemented with the microparticulate systems according to claim 1.

14. Animal feeds supplemented with the microparticulate systems according to claim 1 and non-encapsulated antioxidants.

15. A production process for providing microparticulate systems, the production process comprising the following steps:
a) preparing a solution, suspension or emulsion comprising at least one biocompatible and biodegradable polymer and one anionic, cationic, amphoteric or non-ionic surfactant;
b) solubilizing or dispersing at least one biologically active substance in the solution, suspension or emulsion from step a) thus providing a mixture A;
c) preparing an aqueous solution of at least one monovalent metal ion salt of a biocompatible and biodegradable polymer thus providing a mixture B;
d) adding the mixture B from step c) to the mixture A from step b) thus providing a further mixture;
e) preparing a solution or a dispersion of at least one gastroresistant and enterosoluble polymer thus providing a mixture C);
f) adding the mixture C from step e) to the further mixture from step d) to provide a solution or suspension; and
g) nebulising or extruding the solution or suspension from step f) into an aqueous solution of a soluble divalent or trivalent inorganic ion salt; or
h) as an alternative to step g), nebulising and drying the solution or suspension from step f) by means of a spray-dryer.

16. The production process according to claim 15, wherein said mixture A is a solution, suspension or emulsion comprising at least one biocompatible and biodegradable polymer, one anionic, cationic, amphoteric or non-ionic surfactant and at least one biologically active substance.

17. The production process according to claim 16, wherein the mixture A obtained from step b) is an emulsion or a suspension which is obtained by dissolving an equal amount of a biocompatible and biodegradable polymer and an anionic, cationic, amphoteric or non-ionic surfactant in distilled water, the quantities of the polymer and the surfactant being, respectively, comprised of between: 0.1% and 50% w/v.

18. The production process according to claim 15, wherein step b) is performed by adding to the solution prepared from step a) the at least one biological active substance with constant stirring until a stable solution, suspension or emulsion is obtained (mixture A), and wherein the quantity of the at least one biologically active substance added being comprised of between 0.1% w/v and 50% w/v.

19. The production process according to claim 15, wherein said mixture B is an aqueous solution of at least one monovalent metal ion salt of a biocompatible and biodegradable polymer.

20. The production process according to claim 15, wherein said mixture C is a buffer solution at a pH of between 5 and 9, and comprises a gastroresistant and enterosoluble polymer in a quantity of between 10% and 30% w/v.

21. The production process according to claim 15 wherein the mixture B is added to the mixture A in a volumetric ratio of 1:2, and the mixture thus obtained is added to mixture C in a volumetric ratio of 3:1.

22. The production process according to claim 15, wherein nebulizing the solution or suspension of step f) is performed in step g) through orifices, nozzles, or needles with dimensions between 10 μm and 5000 μm, and extrusion occurs by means of automated, semi-automated microencapsulators, peristaltic, piston or other pumps, or using a manually activated and/or automatic syringe operating at such a speed to produce 10 to 250 drops/minute.

23. The production process according to claim 15, wherein the aqueous solution of a soluble divalent or trivalent inorganic ion salt of step g) is an aqueous solution of calcium, barium, strontium, zinc, aluminium, iron or chromium chlorides.

24. The production process according to claim 15, wherein the aqueous solution of a soluble inorganic ion salt of step g) has a concentration of between 0.1 and 2.0 M.

25. The production process according to claim 15, additionally comprising the step of cross-linking the outer surfaces of the microparticulate systems through the use of cross-linking agents.

26. The production process according to claim 25, wherein said cross-linking agents are in aqueous solutions at concentrations between 0.01 and 5% w/v.

27. The production process according to claim 15, additionally comprising the step of lyophilizing said microparticulate systems.

28. The production process according to claim 15, wherein binding agents and other excipients are added to the solution or suspension from step f) to give a wet mass which is extruded by means of a granulator, to give spheroidal granules.

29. The production process according to claim 28, wherein said spheroidal granules have a granulometric distribution comprised of between 50 and 1000 microns.

30. The production process according to claim 28, wherein said spheroidal granules are coated with a gastroresistant and enterosoluble polymer.

31. The microparticulate systems according to claim 1, having a diameter comprised between 3 and 100 microns.

32. The method according to claim 6, wherein said animals include young of porcines, bovines, caprines, ovines, equids, canids, felines, camelids, lagomorphs, rodents and other mammals including humans, fowl and fish.

33. The process according to claim 16, wherein step b) is performed by adding to the solution prepared from step a) the at least one biologically active substance with constant stirring until a stable solution, suspension or emulsion is obtained (mixture A), and wherein the quantities of said polymer and said surfactant are comprised of between 0.4% and 30% w/v.

34. The production process according to claim 15, wherein said mixture C is a buffer solution at a pH of between 7 and 8, and comprises a gastroresistant and enterosoluble polymer in a quantity of between 10% and 30% w/v.

35. The production process according to claim 15, wherein said mixture C is a buffer solution at a pH of between 5 and 9, and comprises a gastroresistant and enterosoluble polymer in a quantity of between 15% and 25% w/v.

36. The production process according to claim 15, wherein said mixture C is a buffer solution at a pH of between 7 and 8, and comprises a gastroresistant and enterosoluble polymer in a quantity of between 15% and 25% w/v.

37. The production process according to claim 15, wherein nebulizing the solution or suspension of step f) is performed in step g) through orifices, nozzles, or needles with dimensions between 300 μm and 2000 μm and extrusion occurs by means of automated, semi-automated microencapsulators, peristaltic, piston or other pumps, or using a manually activated and/or automatic syringe operating at such a speed to produce 10 to 250 drops/minute.

38. The production process according to claim 15, wherein nebulizing the solution or suspension of step f) is performed in step g) through orifices, nozzles, or needles with dimensions between 10 μm and 5000 μm, and extrusion occurs by means of automated, semi-automated microencapsulators, peristaltic, piston or other pumps, or using a manually activated and/or automatic syringe operating at such a speed to produce 20 to 120 drops/minute.

39. The production process according to claim 15, wherein nebulizing the solution or suspension of step f) is performed in step g) through orifices, nozzles, or needles with dimensions between 300 μm and 2000 μm, and extrusion occurs by means of automated, semi-automated microencapsulators, peristaltic, piston or other pumps, or using a manually activated and/or automatic syringe operating at such a speed to produce 20 to 120 drops/minute.

40. The production process according to claim 15, wherein the aqueous solution of a soluble inorganic ion salt of step g) has a concentration of between 0.2 and 0.8 M.

41. The production process according to claim 25, wherein said cross-linking agents are protamine sulphate or phosphate, poly-L-lysine hydrobromide, polyvinylamine, or chitosans.

42. The production process according to claim 28, wherein said spheroidal granules have a granulometric distribution of between 150 and 500 microns.
